(19) United States
(12) Patent Application Publication
(10) Pub. No.: US 2009/0182309 A1
(43) Pub. Date: Jul. 16, 2009

(54) MEDICAL FLUID COUPLING PORT WITH GUIDE FOR REDUCTION OF CONTAMINATION

(75) Inventor: Matthew K. Muffly, Enfield, NH (US)

Correspondence Address: LOGINOV & ASSOCIATES, PLLC, 10 WATER STREET, CONCORD, NH, 03301, US

(73) Assignee: DARTMOUTH-HITCHCOCK CLINIC, Lebanon, NH (US)

(21) Appl. No.: 12/351,508

(22) Filed: Jan. 9, 2009

Related U.S. Application Data
(60) Provisional application No. 61/010,749, filed on Jan. 11, 2008.

(51) Int. Cl. A61M 39/10

(52) U.S. Cl. 604/535

(57) ABSTRACT

This invention provides a female medical coupling port with an integrated port guide to enable more accurate and precise coupling of a male port coupling (such as the cannula of a syringe) and to prevent port exposure to non-sterile objects. The male and female ports can be arranged according to standard dimensions for male and female luer taper fittings recognized by ANSI and by ISO. This guide-shielded port is usable with the standard ANSI and ISO male cannula widely used in the medical field. In an embodiment the female port is used in medical fluid systems to receive a blunt male cannula, such as those found in the luer lock fitting of needle-less syringes and IV tubing systems to establish a mechanical coupling. Female ports allow coupling of devices (e.g. syringes and IV tubing) to a variety of medical applications including stopcocks, minimum fluid displacement medical couplings, female-to-female adapters, port dead-end caps, IV extension sets, pressure-monitoring devices, etc. The port guide can be constructed as a unitary part of the port, or can be a retrofittable structure that is either snapped into place on, for example, a female port stem, or slid onto a port, such as a minimum displacement fluid coupling. Appropriate drain ports can be provided in the port guide to prevent capture of excess fluid.

### RELATED APPLICATIONS

This application claims the benefit of U.S. Provisional Application Ser. No. 60/010,749, filed Jan. 11, 2008, entitled MECHANICAL COUPLING PORT WITH GUIDE FOR REDUCTION OF CONTAMINATION, the entire disclosure of which is herein incorporated by reference.

### FIELD OF THE INVENTION

This invention relates to medical luer lock fluid couplings or ports, and more particularly to male-female threaded fluid couplings constructed in accordance with ANSI standards.

### BACKGROUND OF THE INVENTION

Fluid systems are a key part of current medical treatment. Fluid systems are used to deliver intravenous (IV) medications, blood and blood components, nuclear medicine agents, and a variety of other liquids/fluids. Fluid systems are also used as the transport conduits for blood and body-fluid circulation equipment including transfusion apparatus, blood filters and warmer mechanisms, and blood dialysis units. The elements of a medical fluid system include a variety of conduits (e.g. flexible polymeric tubing), subcutaneous injection devices (catheters, needles, etc.), valves (e.g. stopcocks), storage and delivery devices (e.g. syringes, IV fluid bags, fluid pumps, etc.), and fluid couplings (e.g. male and female ports) for interconnecting the components of the system. In particular, fluid couplings for medical applications are designed to be easy to connect and disconnect, non-leaking, and manufactured from materials (e.g. transparent, translucent and opaque polymers) and processes (e.g. injection molding, extrusion, etc.) that contemplate disposability after use. A ubiquitous medical fluid coupling system uses threaded female ports and male couplings that engage in a “luer taper” relationship. The parameters and performance of this coupling system is particularly specified under American National Standards Institute (ANSI) standard ANSI/HIMA MD70.1, and also under the similar International Standards Organization (ISO) standard ISO 594. As described in further detail below, this system employs a female port having a proximal end in connection with a fluid system component (tubing, stopcock body, etc.) and a short external thread section on the opposing distal end. The inner surface of the distal end is formed with a somewhat tapered frustoconical shape, which is adapted to receive and seal against the distal end of a conforming male tapered or frustoconical coupling. The opposing proximal end of the male coupling is also interconnected with a fluid system component (tubing, syringe body, etc.). The male and female coupling ports are locked into a fluid-tight and air-tight relationship by an internally threaded nut or axial portion that rotates freely on a flange of the male coupling. Appropriate rotation of the axial portion with respect to the external thread section on the female port drives the male coupling axially into firm engagement with the female port with the two mating elements in a wedged-together relationship due to their respective, conforming tapers.

Although fluid system coupling ports are manufactured and delivered in sterile condition, the problem of fluid system bacterial contamination is well-described in the medical literature. See by way of background Mermel, L., Prevention of Intravascular Catheter-Related Infections, Annals of Internal Medicine 2000; 132:391-402; O'Grady, N. et al. Guidelines for the prevention of intravascular catheter-related infections. Centers for Disease Control and Prevention. MMWR Morb Mortal Wkly Rep 2002; 51(RR-10): 1; Pittet, D., Tarara, D. and Wenzel, R. P., Nosocomial Bloodstream Infection in Critically Ill Patients, JAMA 1994; 271, 1598-1601; Edgeworth, J., Treacher, D. and Eykyn, S., A 25-year Study of Nosocomial Bacteremia in an Intensive Care Unit, Crit. Care Med. 1999; 27:1421-1428; and Laupland, K. B., Zygun, D. A., Davies, D., et al., Population-based Assessment of Intensive Care Unit-acquired Bloodstream Infection in Adults: Incidence, Risk Factors, and Associated Mortality Rate, Crit. Care Med., 2002; 30:2462-2467.

If the port of an intravenous fluid system becomes contaminated with bacteria, the sterility of the entire fluid system is compromised and provides a direct, intravenous, route for introduction of harmful bacteria into the patient. As reported in the above-referenced background publications, there are an average of 5.3 hospital-acquired bloodstream infections per 1000 catheter days in the intensive care unit. Each hospital-acquired bloodstream infection is associated with an approximate mortality rate of 10-35%. Additionally, hospital-acquired bloodstream infections are associated with longer hospitalizations, and impose a significant economic burden. In one current estimate, each hospital-acquired bloodstream infection costs $34,508-$56,000, resulting in an annual cost of $296 million to $2.3 billion annually. An estimated 250,000 cases of hospital-acquired bloodstream infections occur yearly in the United States. Alarmingly, hospital-acquired bloodstream infections have become more frequent, according to one 25-year study referenced above, most likely as a result of the increased use of intravascular catheters, which are the most common source of bloodstream infection. Clearly, contaminated intravenous systems which result in bloodstream infections cause significant mortality, increased length of hospital stay and a considerable economic burden.

The existing design of female medical coupling ports may, in fact, increase the risk of hospital-acquired bloodstream infections. By way of background, reference is now made to FIGS. 1 and 2, which respectively show a perspective and side view of a conventional three-port, four-way stopcock 100 containing two female ports 110, 112 and one male coupling port 114, constructed in accordance with the above-described ANSI or ISO standard for a luer taper system having a threaded “luer lock” arrangement according to the prior art. An exemplary version of this stopcock is shown and described in U.S. Pat. No. 6,418,966, entitled STOPCOCK FOR INTRAVENEOUS INJECTIONS AND INFUSION AND DIRECTION OF FLOW OF FLUIDS AND GASSES, by George Loo, the teachings of which are incorporated herein by reference as useful background information. The stopcock 100 includes a main housing or body 120 that, like other medical fluid system fittings to be described herein, can be constructed from a biocompatible polymer, such as polycarbonate, acrylic, polyvinylchloride (PVC) or acrylonitrilebutadienestyrene (ABS), using injection molding or another suitable construction technique. The body includes a central chamber 120 that is connected by passages (not shown) to each port 110, 112, 114. The passages are sealed and/or interconnected to allow fluid flow by one or more channels formed through a core (not shown) that is rotatably mounted in the chamber 120. The core's passages can be rotated to align with the passages of ports to interconnect the flow between selected ports. Alternatively, the core can be rotated so that passages are sealed with respect to each other, thereby stopping fluid flow through the stopcock 100. The core is rotated (curved double arrow 210) using a lever assembly 130 that includes an extended lever 132. The lever 132 is adapted to be engaged by one or more fingers of the practitioner, and thereby rotates the core about a rotation axis 220 with respect to the chamber 120 to achieve the desired flow setting.

Notably, the depicted female ports 110, 112 include the axially short external thread section 134, 136, respectively, surrounding a tapered female port orifice/passage 140, 142 (also shown in phantom in FIG. 2). The male coupling port 114 in this example includes an internally threaded locking sleeve (or nut) 150 seated upon the male coupling 152. As shown further in FIG. 2, the taper angle TA conforms to the taper of the female port orifice (140, 142) allowing male luer fittings and female luer firings to be nested (generally wedged together) coaxially in a sealed relationship. In this example, the internally threaded (internal threads 156) locking sleeve 150 of the male coupling port 114 rotates freely about the male coupling 152, but is restrained from slipping axially off the coupling by a raised ring 230 or another restraining member. In FIG. 2 the internally threaded locking sleeve 150 is shown in phantom, as it can be omitted in so-called “luer slip” arrangements in which the male and female members are axially pressed onto each other and secured in a fluid-tight relationship by a fiction fit. In such an arrangement, the thread section of the female member can be omitted. In other arrangements, the internally threaded locking sleeve can be fixedly (non-rotatably) secured to the male coupling. Such an arrangement is common where the proximally connected element has increased rotatability, such as where the male port coupling is applied to an end of an elongated, flexible tubing.

Reference is now made to FIG. 3, which shows the exemplary stopcock 100 in use with interconnected fluid system components 302 attached to the female port 114 by a threaded male coupling 304. The stopcock 100 is manipulated by a practitioner whose hand 310 grasps the rotatable lever assembly 130, 132 with his or her thumb 312 and forefinger 314. Note that the hand 310 is ungloved, which is typical in many procedures involving the use of a fluid system (often due to the greater dexterity required to manipulate fluid system components). Despite the practitioner's proper and diligent efforts to scrub hands with disinfectant, substantial live microbiological residue usually remains thereon, and may easily become deposited on the distal tip (and threads 136) of the female port 112 as the fingers glance and contact it while moving (double arrow 320) the lever 132 to a new position (as shown in phantom). Many other opportunities to contaminate some or all of the fluid couplings and associated components also exist.

For example, to place male threaded/locking coupling (syringe, tubing end, etc.) in engagement with the port 112, a series of steps must be carefully taken to maintain sterility. First, a sterile cap 330 having a stoppered, threaded male end 332 and a (knurled) gripping surface 334 is unscrewed from the port 112 and placed in a sterile location as shown. Next, as depicted in FIG. 4, the practitioner manipulates a syringe 400 with an associated male cannula, which in this example is the male luer coupling 410 with an internal thread 450 and male taper luer 460 mounted on the distal end of a syringe barrel 430 having a proximal plunger 432. The syringe 400 is lowered (arrow 440) by the practitioner's hand 310 to place the distal male coupling 410 into alignment and engagement with the female port 112 and its associated female taper luer hole 142 and external thread 136. As shown in FIG. 5, once the distal male coupling 410 is properly aligned, the syringe barrel 430 is then twisted (arrow 510) to engage the distal male coupling's internal thread 520 until it is firmly and securely locked onto the female port 112 in a fluid-tight seal. The practitioner can then deliver or withdraw a measured volume of fluid, medication, etc. by axially depressing or withdrawing (double arrow 520) the plunger 432 with respect to the syringe barrel 430. Thereafter, the practitioner twists off and disconnects the syringe coupling 410 from the female port, and reconnects the cap 330 so as to prevent inadvertent leakage of fluid or subsequent contamination of the thread 136 or female luer taper orifice 142. The recapped port 112 is shown in FIG. 6. Note that the presence of a standard (though blocked) female luer lock fitting 370, with proximal external thread 372 allows a number of similar caps 330 to be stacked male-to-female end for “safe-keeping” as shown. One may even place the syringe coupling (410) or other working male luer coupling at the proximal end of this stacking arrangement 610 as also shown.

Note, as used herein, terms such as “proximal” and “distal” shall refer to the relative direction of a component in the fluid system with respect to the practitioner and/or patient. The component side facing the practitioner, and into which an injection, etc. is directed, is typically “proximal”, while the component side facing the patient, or another downstream device is “distal”. However, these definitions are only conventions used to provide relative locations of a component. Likewise term such as “axial”, “up”, “down”, etc. are conventions and not absolute directions.

The practitioner repeats these steps multiple times (e.g. for each medication that is delivered or fluid administered), thereby significantly increasing the risk of port contamination and patient infection due to the ever-present risk that non-sterile hands or implements will contact the port 112. The constant handling, putting aside, and possible stacking of the small cap(s) poses another risk of port contamination. Adding to the risk of contamination, the practitioner must manually steady the sterile port with respect to the male coupling, in most instances, to establish the connection. In so doing, the practitioner's fingers may inadvertently touch the sterile port, or the male luer taper may slip off the sterile female port and touch against the fingers that are stabilizing the stopcock 100. This renders the syringe and the potentially costly medication therein useless (or hazardous/fatal if used). As described above, for example using a stopcock, the location of the lever essentially invites finger-contact with the port. In addition, fluid ports (capped and uncapped) often lie casually against the patient's gown and/or skin between uses—and may even become dragged onto non-sterile surfaces, sometimes with threads exposed to these surfaces.

As described above, the problem of fluid system contamination is well-known in the medical literature. While proper hand hygiene must be practiced to reduce hospital-acquired infections, medical device innovation may also reduce this risk. One device which can potentially reduce contamination of ports is taught in U.S. Pat. No. 5,730,418, entitled MINIMUM FLUID DISPLACEMENT MEDICAL CONNECTOR, by Feith, et al., the teachings of which are incorporated herein by reference as useful background information. The minimum fluid displacement medical coupling described therein eliminates the need for capping and recapping the female port to avoid inadvertent fluid loss therethrough by providing a self-sealing proximal female taper luer coupling tip that is adapted to connect with a standard threaded (locking) male taper luer coupling. By way of example, FIG. 7 shows commercially available version of the minimum fluid displacement medical coupling 700. The coupling 700, also commonly termed a “clave”, consists of a housing 710 that includes a proximal female taper luer port end 712 with standard external threads 714 adapted to engage the locking sleeve of a male taper luer lock coupling. On the distal end of the housing 710 is a male taper luer lock coupling 720 having an internal thread 722 and male taper luer 724 with a central passage 726 that allows fluid-flow into an interconnected conventional female luer taper port (such as the fluid entry port of a stopcock, as described below). The female port 712 and male port 720 are in fluid communication via the inner chamber 730 of the housing 710. The female port is normally sealed by a soft polymeric (rubber, for example) plug 740 that is biased into the inner wall of the port opening 742 into a sealed relationship therewith. The proximal biasing force is generated by an integral/unitary spring body 744 (defining a bellows shape) with an opposing base end 746 that rides on a central, vented guide 746 adjacent to the male port 720. In alternate embodiments, a separate compression spring can be used to generate the proximal, sealing bias force. When, as described further below, the plug 740 is biased distally (arrow 750) by a fluid system taper luer end, it opens a channel between the port opening 742 and the inner chamber 730, and thereby allows fluid to travel between the female port 712 and the passage 726 of the male port 720 of the coupling via the inner chamber 730. Upon removal of the locked-on male taper luer from the biased plug end 712 of the coupling 700, the plug moves back into a sealing position against the inner wall 742 of the female port, thereby preventing fluid loss.

While this coupling 700 effectively avoids unwanted leakage or loss of fluid from the proximal female port 712, this coupling, however, does not improve the precision and accuracy of making medical connections, nor does this coupling prevent inadvertent port contact with non-sterile objects or body parts. For added protection a separate (also potentially contaminated) cap must be applied to the female port. This particular exemplary minimum displacement fluid coupling also does not provide a stopcock mechanism for variable direction of fluid flow, but must be applied to the port of a conventional stopcock.

Medical device innovation aimed at improving the precision and accuracy of making connections and reduction of contact with non-sterile objects may reduce contamination of fluid systems and ultimately decrease the number of hospital-acquired bloodstream infections. Accordingly, it is highly desirable to provide a system that functions to improve the precision and accuracy of establishing a medical fluid coupling and that protects the sterile nature of the fluid port from contact with non-sterile objects with the ultimate goal of reducing patient infections. This system should be fully compatible with existing luer-taper and similar friction-fit and threaded coupling systems and should integrate with either conventional ports or minimum displacement fluid coupling ports. The system should also be applicable to a variety of medical fluid system components and couplings including stopcocks of various types, IV interfaces/spike connections, injection ports, tubing couplings and adapters, and the like.

### SUMMARY OF THE INVENTION

This invention overcomes the disadvantages of the prior art by providing a female medical coupling port with an integrated port guide to enable more accurate and precise coupling of a male port coupling (such as the cannula of a syringe) and to prevent port exposure to non-sterile objects. The male and female ports can be arranged according to standard dimensions for male and female luer taper fittings recognized by ANSI and by ISO. Thus, this guide-shielded port is usable with the standard ANSI and ISO male cannula widely used in the medical field. In an embodiment, the female port is used in medical fluid systems to receive a blunt male cannula, such as those found in the luer lock fitting of needle-less syringes and IV tubing systems to establish a mechanical coupling. Standard male luer lock fittings have a male luer taper surrounded by a threaded locking collar or sleeve which enables coupling with female ports. Female ports allow coupling of devices (e.g. syringes and IV tubing) to a variety of medical applications including stopcocks, minimum fluid displacement medical couplings, female-to-female adapters, port dead-end caps, IV extension sets, pressure-monitoring devices, epidural or intrathecal catheter tubing, etc. The port guide can be constructed as a unitary part of the port, or can be a retrofittable structure that is either snapped into place on, for example, a female port stem, or slid onto a port, such as a minimum displacement fluid coupling (clave).

In an illustrative embodiment, the medical fluid coupling comprises a female port of a first medical fluid system component including a proximal port end that is constructed and arranged to sealingly engage a male port coupling. A port guide defines a sidewall that surrounds the female port and extends from a distal end of the female port to a proximal guide end. The proximal guide end is open to receive the male port coupling and located proximally at a spacing from the proximal port end, so as to prevent contaminating contact with the female port and aid to in guiding the male port coupling into alignment and engagement with the proximal port of the female port. The female port can comprise a female luer taper port and the male port can comprises a male luer taper port in which the proximal port end can define an external locking thread and the male port defines an internally threaded collar or sleeve, surrounding a luer taper connector tip. The threaded collar or sleeve is constructed and arranged to threadingly engage the external thread. The luer taper geometry of the male/female ports and the thread dimensions can be in accordance with ANSI and/or ISO specifications.

In an illustrative embodiment, the female port includes a housing on a distal region thereof comprising a minimum fluid displacement coupling and the proximal port end includes a movable self-sealing plug therein. The guide can be adapted to removably slide onto the housing, or can be formed unitarily with the coupling. In another illustrative embodiment, typically applicable to ports that include a stem and threaded proximal end, the port guide can include a pair of axially spaced apart resilient central supports, such as O-rings, having an un-flexed inner diameter equal to or slightly less than the outer diameter of the stem. The O-rings are adapted to flexibly pass over the threaded portion and captures the distal stem of the port-thereby providing a retrofittable structure that can be used with the conventional ports of stopcocks and other fluid system components. Appropriate drain ports can be provided to channel fluid away from the proximal region above the O-rings/resilient central supports. Other attachment and fixing mechanisms, such as the use of a guide with clamshell halves or a separate attachable mounting base can be employed in alternate embodiments to provide an attachable/retrofittable port guide to a port structure.

In various embodiments herein, the port guide defines, at a proximal region thereof, an outward taper in the proximal direction.

### BRIEF DESCRIPTION OF THE DRAWINGS

The invention description below refers to the accompanying drawings, of which:

FIG. 1, already described, is a perspective view of a conventional three-port four-way stopcock including male and female taper luer ports according to the prior art;

FIG. 2, already described, is a side view of the stopcock of FIG. 1;

FIG. 3, already described, is a diagram showing the stopcock of FIG. 1 interconnected with a medical fluid system, and the lever thereof manipulated by the hand of a practitioner in a manner that risks microbiological contamination of a fluid-entry port;

FIG. 4, already described, is a diagram showing the stopcock and fluid system of FIG. 3, and a syringe with a conventional male taper luer coupling being brought into connection with a female luer taper port of the stopcock by the hand of a practitioner in a manner that risks microbiological contamination of a fluid-entry port;

FIG. 5, already described, is a diagram showing the stopcock and fluid system of FIG. 3, with the male taper luer coupling of the syringe in connection with the female luer taper port of the stopcock;

FIG. 6, already described, is a diagram showing the stopcock and fluid system of FIG. 3, with the male taper luer coupling of the syringe disengaged from the female luer taper port of the stopcock and a plurality of caps stacked onto the female luer taper port of the stopcock;

FIG. 7, already described, is a side cross section of a minimal fluid displacement coupling including a self-sealing, minimum fluid displacement medical coupling with opposing male and female luer taper lock couplings according to the prior art;

FIG. 8 is a top view of a three-port, four-way stopcock including a threaded, locking female taper luer port with a contamination-reducing port guide according to an illustrative embodiment of this invention;

FIG. 9 is a partial cross sectional perspective view of a three-port four-way stopcock including a pair of threaded, locking female taper luer ports each with a contamination-reducing port guide according to an illustrative embodiment of this invention;

FIG. 10 is a diagram showing the illustrative stopcock of FIG. 8 interconnected with a medical fluid system, and a syringe with a conventional male taper luer coupling being brought into connection with the female luer taper port with the contamination-reducing port guide;

FIG. 11 is a diagram showing the illustrative stopcock of FIG. 8 interconnected with a medical fluid system, and the syringe coupled with the female luer taper port with the contamination-reducing port guide;

FIG. 12 is a diagram showing the illustrative interconnected stopcock of syringe arrangement of FIG. 11 with the stopcock manipulated by one hand of the practitioner while another hand operates the syringe plunger with the port area protected from contamination by the port guide;

FIG. 13 is a diagram showing the illustrative stopcock of FIG. 8 interconnected with the medical fluid system, with a smaller-diameter syringe coupled with the female luer taper port with the contamination-reducing port guide;

FIG. 14 is a diagram showing the illustrative stopcock of FIG. 8 interconnected with the medical fluid system, with a larger-diameter syringe coupled with the female luer taper port with the contamination-reducing port guide;

FIG. 15 is a partial perspective view of a series of interconnected stopcocks that are part of a fluid system resting on a patient's gown while receiving treatment therefrom, with reduced risk of contamination from the environment;

FIG. 16 is a perspective view of an illustrative three-port, four-way stopcock including a threaded, locking female taper luer port with a contamination-reducing port guide being interconnected with a conventional male taper luer connector with threaded locking sleeve;

FIG. 17 is a perspective view of a threaded, locking female taper luer port mounted as an end connector on a flexible tubing, and including a contamination-reducing port guide being interconnected with a conventional male taper luer connector with threaded locking sleeve, according to an illustrative embodiment;

FIG. 18 is a perspective view of an IV bag spike connection including a threaded female luer taper coupling port according to the prior art;

FIG. 19 is a top view of the IV bag spike of FIG. 18;

FIG. 20 is a perspective view of an IV bag spike including a threaded, locking female taper luer port with a contamination-reducing port guide according to an illustrative embodiment of this invention;

FIG. 21 is a top view of the IV bag spike of FIG. 20;

FIG. 22 is a diagram showing the insertion of the IV bag spike of FIG. 19 into an exemplary IV bag and associated interconnection of the female taper luer port of the spike with a male taper luer coupling on an IV system tubing;

FIG. 23 is a side cross section of a minimum fluid displacement coupling including a contamination-reducing port guide according to an illustrative embodiment of this invention;

FIG. 24 is a side cross section showing the illustrative minimum fluid displacement coupling with port guide of FIG. 23 interconnected with an exemplary syringe having a threaded male taper luer coupling;

FIG. 25 is an exploded perspective view of a minimum fluid displacement coupling and associated port guide according to an illustrative embodiment shown mounted on the female luer taper port of an exemplary conventional three-port, four-way stopcock and interconnected medical fluid system;

FIG. 26 is a perspective view of the minimum fluid displacement coupling and port guide of FIG. 25, shown assembled and mounted on the female luer taper port of an exemplary conventional three-port, four-way stopcock;

FIG. 27 is a diagram showing the illustrative stopcock and minimum fluid displacement coupling with port guide according to FIG. 26 interconnected to the medical fluid system, with a smaller-diameter syringe coupled thereto;

FIG. 28 is a diagram showing the illustrative stopcock and minimum fluid displacement coupling with port guide according to FIG. 26 interconnected to the medical fluid system, with a larger-diameter syringe coupled thereto;

FIG. 29 is a side cross section of a medical fluid system tubing having a minimum fluid displacement coupling and port guide attached thereto, and interconnected with a threaded male luer taper coupling attached to another medical fluid system tubing, according to an illustrative embodiment;

FIG. 30 is a perspective view of an attachable port guide for use with conventional threaded female luer taper coupling ports according to an illustrative embodiment; and

FIG. 31 is a fragmentary perspective view of the illustrative attachable port guide of FIG. 30 installed on a threaded female luer taper port of an exemplary three-port, four-way stopcock and having attached thereto an exemplary syringe.

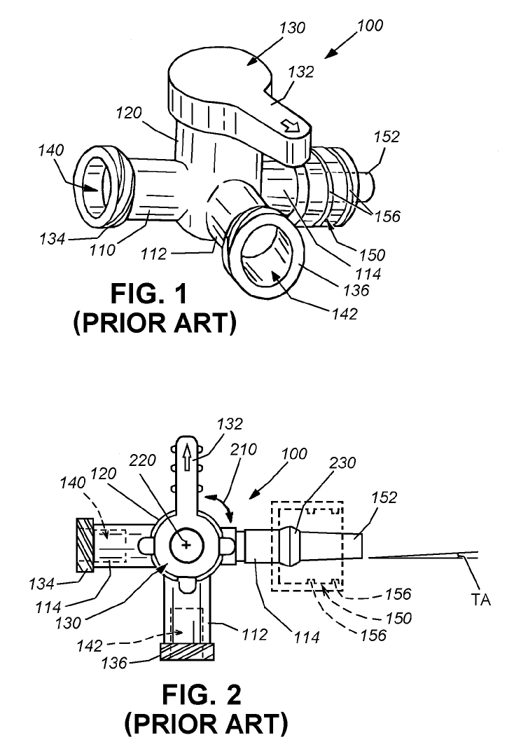

### DETAILED DESCRIPTION

FIG. 8 is top view of a three-port, four-way stopcock 800 with a conventional rotating (double curved arrow 812) lever assembly 810 having a conventional externally threaded female luer taper port 820 and opposing male luer taper port 822 (with threaded locking sleeve omitted). A third, externally threaded female luer taper port 830, typically positioned at a syringe-coupling location, is also provided in accordance with an illustrative embodiment. This port 830 includes a stem 832 extending proximally from the stopcock's central chamber 840 (which houses the core of the lever assembly 810). The stem 832 ends in a conventional, axially shortened external luer lock thread 850. The thread surrounds a luer taper orifice and passage 852 (shown in phantom) as described above.

Notably, the female port stem 840 is surrounded by a port guide 860 in accordance with an illustrative embodiment. The port guide 860 in this embodiment is constructed from a polymer that is shown as transparent. In alternate embodiments, the port guide and/or other parts of the stopcock can be constructed from translucent or opaque materials. Note that where a polymer is used to construct the port guide and/or other portions the fluid system component it can be of an antimicrobial type, including appropriate antibacterial fillers and additives. The port guide extends from the central chamber 840 to a proximal edge 862 residing axially/proximally beyond the proximal end of the port thread 850. This additional distance of proximal extension DPE is highly variable. In an illustrative embodiment it is between approximately 2 and 6 millimeters. As described further below, the distance DPE should be sufficient to provide overlapping coverage for the port/thread's proximal end 854, but not so long as to prevent a conventional male luer taper cannula of a syringe, for example, from fully seating onto the female port. In order to provide clearance from such a male cannula, a radial spacing RS is also established between the maximum outer perimeter of the thread 850 and the inner wall of the port guide 860 in its proximal region 864. This radial spacing RS is sufficient to accommodate the thickness and maximum outer diameter of a conventional male luer lock internally threaded sleeve. In an embodiment, RS is at least approximately 2-5 millimeters. However, this distance is highly variable so long as the distance RS is sufficient to accommodate the thickness and outer diameter of the thickest/largest-diameter diameter male cannula/coupling to be accommodated by the port 830. The proximal region 864 of the port guide 860 is optionally flared to a larger diameter as shown to provide the cannula clearance distance RS in the region of the thread. The clearance (RS) should extend distally (toward the central chamber 810) past the thread 850 by a distance DC that allows the distal tip of the longest locking cannula threaded sleeve to remain unobstructed when the cannula is fully locked onto the port 830. In an embodiment, the distal clearance DC is at least between approximately 4 and 10 millimeters. However, a longer extension distance of the large-diameter region of the port guide is contemplated, and in alternate embodiment, the larger inner port guide diameter can extend to the central chamber. In an embodiment this inner diameter is between at least approximately 9 and 12 millimeters, but larger (or somewhat smaller) port guide inner diameters are expressly contemplated.

Reference is now made to the partial cross-sectional view of a similar stopcock 900 to that (800) shown in FIG. 8. This illustrative stopcock 900 includes a central chamber 910 with rotating lever assembly as described above. It also includes a male luer taper port 922 with internally threaded locking sleeve 924. Notably, this embodiment includes a pair of externally threaded female luer taper ports 930 and 940 each with corresponding, surrounding port guides 950 and 960, respectively. The guides exhibit inner diameters and clearances that are generally in accordance with the dimensions described above.

In this embodiment, the proximal region 952, 962 of each respective guide 950, 960 is provided with a proximally outward flare or taper such that the proximal end 954, 964 is of larger inner diameter than the region adjacent to the port thread 932, 942 is of a slightly smaller diameter. This enhances the ability of the port guide 950, 960 to assist the practitioner in more accurately and precisely aligning a male cannula with the female port by providing, in essence, a funnel effect. The angle of the taper (GTA) with respect to the axial (distal-to-proximal) direction can vary greatly. In an embodiment, the angle GTA is between approximately 2 degrees and 10 degrees. However other taper angle ranges are expressly contemplated. Likewise the flare or taper may be provided only along a portion of the proximal region (e.g. a short funnel end), so long as the more distal remainder of the region provides an inner diameter with needed clearance for the cannula. Alternatively the taper can be carried beyond the proximal region, and optionally to the central chamber or other component base to which the port guide and/or port stem is attached. Furthermore, the taper need not be a single angular dimension (i.e. a frustoconical shape), but alternatively can define a compound angle and/or curvilinear bowl shape. Additionally, the radially directed wall thickness WT of the port guide in any embodiment herein can be highly variable. In an embodiment, the thickness WT is between approximately 0.5 and 3.5 millimeters, but other dimensions are expressly contemplated and should afford sufficient structural strength to the port guide with respect to the material being used to construct it. In various embodiments, the proximal edge and/or another portion of the guide can include one or more strengthening ribs or lips that define thickened portions. For example, as depicted in various embodiments herein, the proximal edge includes a radially thickened lip.

Notably, it is contemplated that the port guide could potentially retain excess fluid from a fluid-delivery or fluid-withdrawal in proximity to the stem and port—potentially contaminating these elements. Thus, the port guides 950, 960 are provide with one or more through-cut drain ports 970 at various locations about the circumference of each guide and at various locations along the length of the guide. These holes are large enough in opening area to rapidly drain any excess fluid captured by the port guide during a procedure, but small enough to prevent infiltration of foreign matter during handling. For example, holes having a diameter of 0.5-1.5 millimeters can be employed in an embodiment. In illustrative embodiments, drain ports 970 can be located as close as possible to the distal base of each guide where the inner diameter of the port guide initially defines an inner hollow region or chamber. Drain ports 970 can also be located at additional locations along the guide's wall to ensure more rapid and efficient draining of fluid when it reaches a heightened level within the space between the guide's inner wall and the port stem. The size, shape, number and position of drain ports are all highly variable. While depicted as rounded holes, the ports can define polygonal slots, elongated grooves, and the like. For example, in an alternate embodiment, the drain ports can define a set of narrow slots located at predetermined positions around the circumference of the port guide extending from the base to a proximal position below the level of the port proximal end. A variety of alternate drain port arrangements are expressly contemplated.

The port guide according to various embodiments herein can be constructed by a variety of techniques, and provided to the underlying female luer taper port in a variety of manners. For example, where the guide is constructed as a separate unit to be subsequently attached to the fluid system component, it can be constructed from extrusion, molding (injection molding, blow-molding, etc.) or machining from solid stock. Such a separate port guide is then attached and permanently or removably adhered to the underlying fluid system component using friction fit, snap fit, adhesives, welding (ultrasonic, for example), fasteners, or other suitable attachment techniques and mechanisms. In other embodiments, in which the port guide is unitary with the underlying fluid system component and port, it can be formed thereon by molding, machining, extrusion (typically in the case of a linear or tubular component), and/or other techniques that facilitate formation of a nested shape with the port guide surrounding, and extending proximally beyond, the proximal end of the female port.

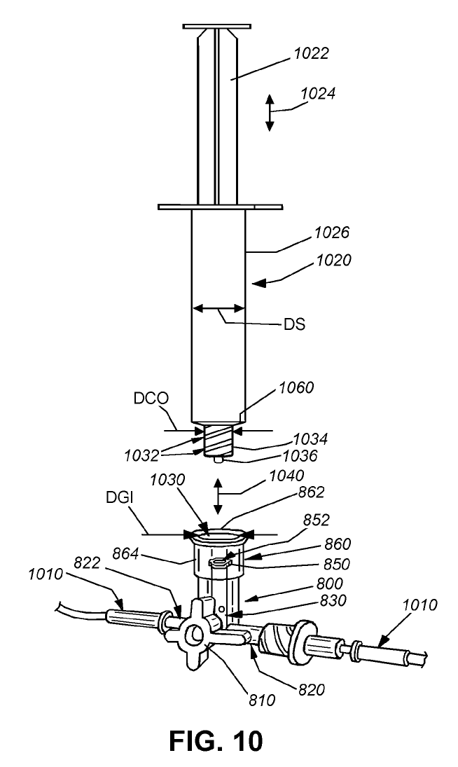

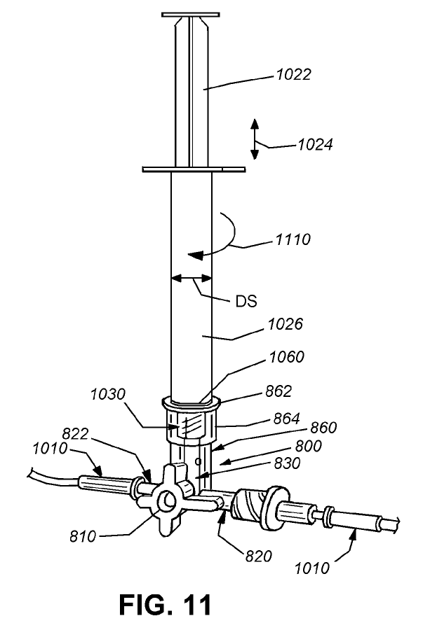

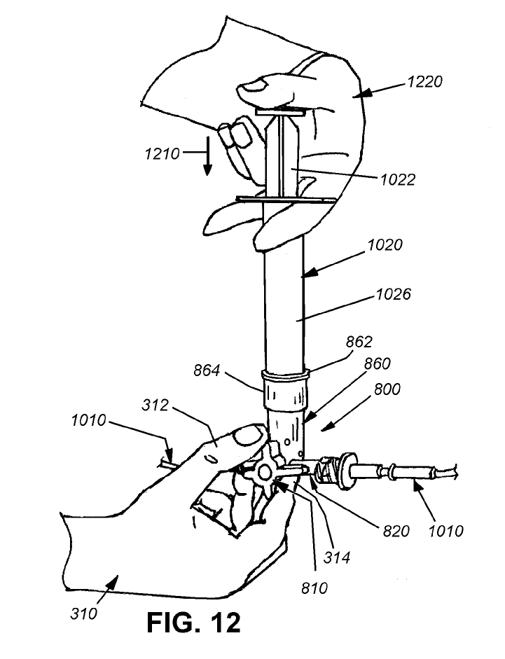

Reference is now made to FIG. 10, which shows the use of the port guide 860 on the above-described stopcock 800 (depicted in an interconnection with a medical fluid system 1010) in conjunction with a conventional injection syringe 1020 with a plunger 1020 movable axially (double arrow 1024) within the syringe barrel 1026 so as to direct or withdraw fluid via the distal cannula 1030. In this example, the cannula is a conventional male luer taper coupling with an internally threaded (threads 1032) outer sleeve 1034 and coaxial, distally projecting male luer coupling 1036. As shown, the practitioner can bring the cannula 1030 into and out of engagement (double arrow 1040) with the port guide opening 1030 and its surrounding proximal edge 862. The increased inner diameter DGI of the port guide's proximal edge 862 relative to the outer diameter DCO of the cannula 1030 assists in guiding the cannula toward the port thread 850, with the male luer taper coupling 1036 being funneled into engagement with the female port orifice/passage 852. Once the cannula 1030 resides within the surrounding guide it will not easily jump out or inadvertently slip onto the practitioner's other hand (which is manipulating the stopcock 800 as shown in FIG. 12), while the barrel 1026 of the syringe 1020 is twisted to lock the threads 550, 1032 into engagement. This fluid-sealed/fluid-tight engagement is shown in FIG. 11 wherein the syringe barrel 1026 has been fully twisted (curved arrow 1110) to sealingly engage the cannula with respect to the female port 830. In this orientation, the cannula is fully, or nearly fully, surrounded by the port guide wall, thereby substantially protecting it from contact or infiltration of contamination. As described above, the proximal extension of the proximal region 864 of the port guide above the proximal end of the port 830 is chosen to ensure that the syringe barrel 1026 is not interfered with by the proximal edge 862 of the guide, regardless of the outer diameter DS of the barrel 1026. Hence the distal shoulder 1060 between the syringe barrel 1026 and the cannula 1030 resides at least slightly spaced-apart from the guide's proximal edge 862 when the cannula is fully tightened onto the female port as shown in FIG. 11. In alternate embodiments, the proximal edge can be sized and arranged to overlap part of the syringe barrel-at least for syringe barrels having a predetermined maximum diameter DS.

It should be clear that the illustrative port guide 860 effectively isolates the port 830 from contamination under a variety of circumstances. Notably, and as shown in FIG. 12, the practitioner's hand 310 can effectively and firmly grasp and manipulate the stopcock, free of the risk of inadvertently contacting a portion of the port. In fact, the port guide 860 defines another convenient gripping surface when administering an injection—as shown, pushing (arrow 1210) the plunger 1022 with the opposing hand 1222—or manipulating the lever assembly 810. When the syringe 1020 is disconnected or removed, the port (830) the proximal end of the port is recessed so that the risk of contact with contaminants is significantly lessened. In fact, the inner diameter of the port guide combined with the distal offset of the port from the proximal edge of the guide may render contact with the port by normal adult fingers nearly impossible. Likewise, even if the port guide's proximal edge is stood on edge against a non-sterile surface, the contamination cannot reach the port. Of course where the edge is exposed to contamination, care should be taken to avoid contacting the cannula with the exterior of the guide. However this is a significantly easier goal to achieve for most practitioners than attempting to align an unguided male cannula on a female port.

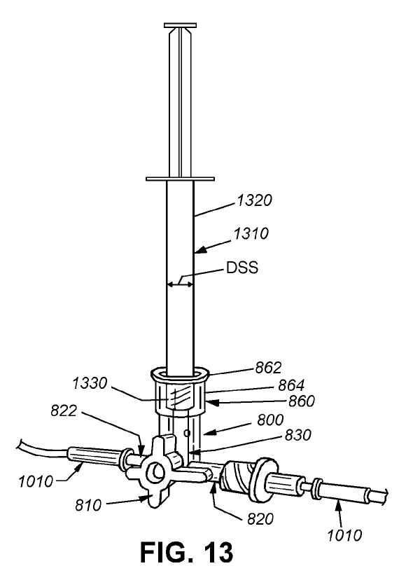

As described above, the proximal region 864 of the exemplary port guide is sized and arranged to accommodate a standard-sized cannula for syringes (and other fluid system components having male couplings) regardless of the external dimensions (diameter DS of the syringe barrel (or other component). With reference to FIG. 13, a syringe 1310 having a small-diameter (DSS) barrel 1320 is shown with its cannula 1330 threadingly engaged to the port 830. The diameter DSS is the same or slightly larger than that of the cannula, and thus, the syringe 1310 passes easily into the proximal region 864 of the port guide 860 with extra clearance room. Nevertheless, the risk of contamination to the port is still significantly reduced, both when the syringe 1310 is engaged and disengaged.

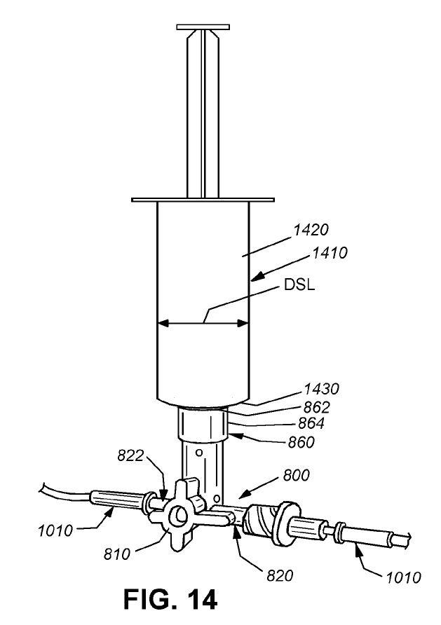

Likewise, as shown in FIG. 14, a syringe 1410 having a large-diameter (DSL) barrel 1420 is shown threadingly engaged to the port. The barrel diameter DSL is significantly larger than that of the cannula (not shown), however, the distal end of all syringes (regardless of the barrel diameter) are generally standardized, conforming to ANSI and ISO measurement standards. Therefore, the larger-diameter syringe will fit free of interference into the proximal region 864 of the port guide 860. The location of the guide's proximal edge 862 combined with the standardization of male luer taper components ensures that the shoulder 1430 between the cannula section of the syringe and the large-diameter barrel remains at leased slightly spaced-apart from the port guide 860 (and proximal edge 862) when the syringe 1410 is fully twisted onto the stopcock female luer taper port. The guide is particularly beneficial in easing the task of guiding and aligning (funneling) a large, and high-volume syringe, which may otherwise prove difficult to manipulate onto the small female port fitting.

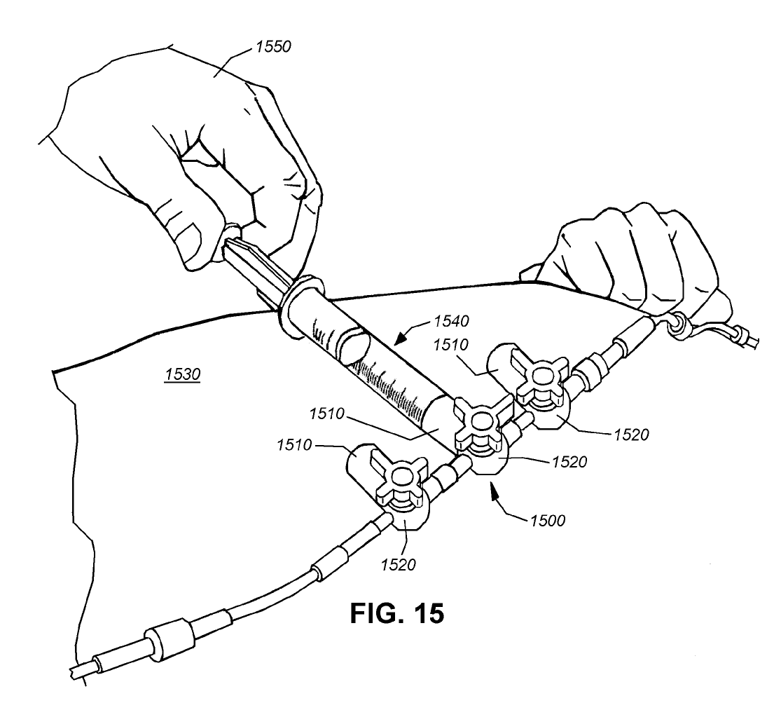

As shown in FIG. 15 the benefits of a medical fluid system 1500 containing port guides 1510 in accordance with an illustrative embodiment become even more apparent. As shown, each interconnected stopcock 1520 in the system 1500 includes a practitioner-accessed port with a guide thereon. As is often typical the stopcocks 1520 rest as a unit on the patients' chest/garment 1530. The practitioner (hand 1550) can administer fluid/medication or withdraw fluid via the syringe 1540 in interconnection with a port of the system 1500 with reduced risk of contamination. When disconnected, the ports are shielded by the guides 1510 from contamination by the patient's garment or skin, or that of surrounding surfaces and persons.

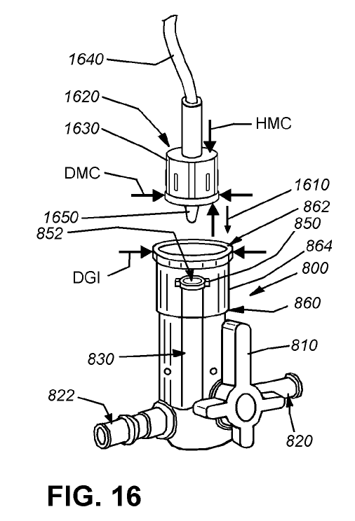

The port guide 860 is sized and arranged to receive a variety of threaded sleeves for male taper luer connectors, as described generally above. With reference to FIG. 16, the above-described stopcock 800 and associated female taper luer port 830 and port guide 860 is adapted to receive (arrow 1610) a conventional male taper luer coupling 1620 with rotating internally threaded sleeve 1630 mounted at the distal end of a conventional flexible medical fluid tubing 1640. The tubing's distal end includes a male luer taper coupling 1650 sized and arranged to sealingly engage the female port orifice 852. The inner diameter DGI of the proximal region 864 of the port guide 864 is greater in diameter that the outer diameter DMC of the threaded sleeve 1630, including any outward protuberances (e.g. grip surfaces, knurling, etc.) thereof. In general, the distal proximal extension (DPE in FIG. 8) of the port guide is selected so that a portion of the sleeve 1630 having an axial height/length HMC remains grippable, even when fully engaged on the thread 850. In alternate embodiments, the height/length HMC can be lengthened, or additional proximal gripping surfaces (for example molded-on or applied tabs or wings) can be provided to proximally extend the gripping surface where the majority of the sleeve is embedded into the guide's proximal region 864 during engagement with the port 830.

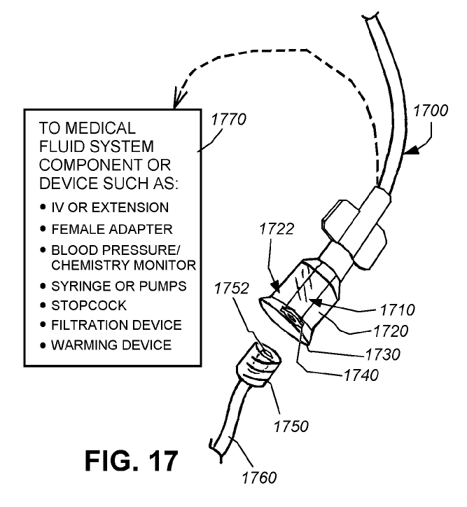

It should be clear that the illustrative port guide in accordance with various embodiments of this invention can be employed with a variety of female luer taper ports, attached to various medical fluid system components. As shown in FIG. 17, a flexible tubing 1700 for a medical fluid system can include a distal end having a female luer taper port 1710 as described generally herein. The port is surrounded by an appropriately sized port guide 1720 of sufficient inner diameter-the size of the inner diameter being in accordance with the dimensions described herein particularly in the proximal region 1722 between the (optionally) flared proximal end 1730 and the area directly distal of the port thread 1740. These dimensions allow the reception and threading engagement of an internally threaded sleeve 1750 of conventional or modified design (e.g. modified to extend axial length). The sleeve in this embodiment is attached to a conventional male luer taper coupling 1752 that is in fluid communication with a second fluid tubing 1760. However, the sleeve 1752 and male luer taper coupling can be attached to any appropriate fluid system component that is desirably connected with the port 1710. Some exemplary components 1770 that can be combined with or substitute for the tubing 1700 are described. These fluid system components (1770) include, but are not limited to IV systems or IV extension sets, female-to-female adapters, fluid/blood pressure and/or fluid/blood chemistry monitors, syringes, pumps and/or other fluid-delivery/withdrawal devices, stopcocks and valves, fluid filtration and fluid warming devices, all defined generally as “fluid handling devices”.

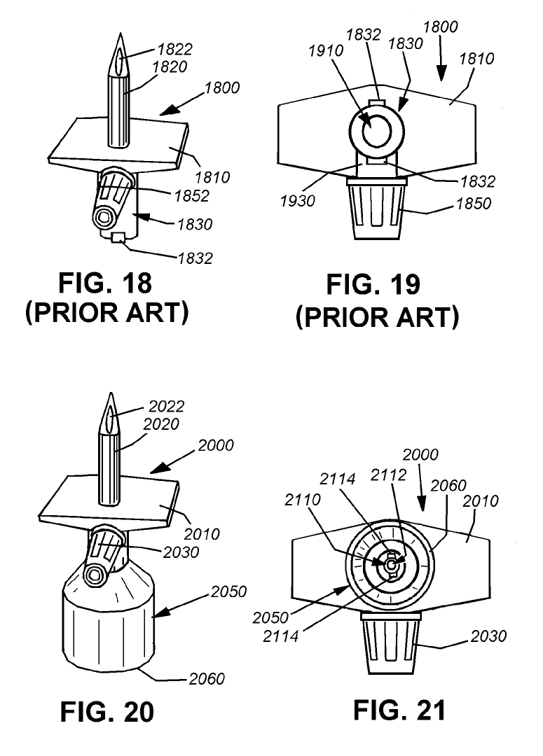

A further use for the port guide according to embodiments of the invention is shown with reference to FIGS. 18-22. A common element in medical fluid systems is an intravenous (IV) bag or container, which can contain any of a variety of medical fluids for administration to the patient by well-known IV infusion procedures. As shown in FIGS. 18 and 19, the fluid interface for an IV fluid bag is the so-called IV spike 1800. The IV spike can also be used to withdraw fluid from a container into a syringe. The spike 1800 consists of a base plate 1810 used for securing the spike against the bag or container (i.e. bottle of medication) (described below), and a sharpened unitary shaft 1820 with a central lumen that passes into a proximal connector 1830. The connector defines a threaded female luer taper coupling in this embodiment. Note that the thread 1832 defines a pair of opposing teeth that are circumferentially interrupted in this embodiment. A substantially circumferentially continuous thread can be provided in alternate examples. At the connector end, the lumen 1822 defines a female luer taper orifice 1910 that is adapted to mate coaxially with a conventional threaded male luer taper coupling and internally threaded locking sleeve. This prior art spike structure, like other unshielded port structures is subject to potential contamination—particularly when reconnected to the fluid system over multiple cycles, but even after a single connection event in which the port 1830 is exposed to contamination. As shown, the spike 1800 can contain a side connection 1930 in fluid communication with the lumen 1822, with the use of air vent 1852 to prevent the creation of a vacuum during the transfer of fluid.

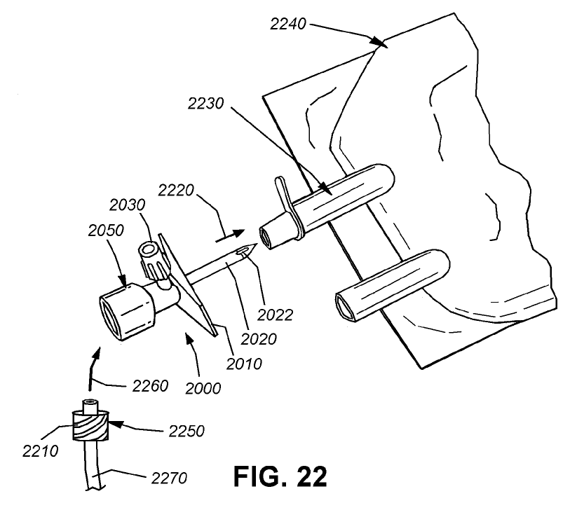

With particular reference to FIGS. 20 and 21, an IV bag or container (e.g. medication bottle) spike connection 2000 according to an illustrative embodiment is detailed in perspective and top views. This spike 2000 includes a base plate 2010 a sharpened shaft 2010 with central lumen 2022 as described above. The lumen 2022 is in fluid communication with the orifice 2112 a female luer taper port 2110 having a proximal thread 2114 for engaging the internal thread of a male luer taper locking sleeve 2210 (FIG. 22). The lumen 2022 is interconnected with an optional side port covered by a cap 2030 in this embodiment. The port 2110 is surrounded by a port guide 2050. The inner dimensions of the port guide 2050 are similar or identical to the embodiments described hereinabove. In general, the proximal end 2060 and adjacent proximal region extend proximally past the port 2110 to fully cover it against inadvertent contact, and defines an inner diameter over an applicable axial distance with respect to the thread 2114 that receives and accommodates a male internally threaded sleeve 2210.

As shown further in FIG. 22, the shaft 2020 of the spike 2000 is inserted (arrow 2220) into a port 2230 of an exemplary IV fluid bag 2240. The internally threaded sleeve 2210 of a male taper luer coupling 2250 is, likewise, interconnected (arrow 2260) to the spike's port (2110) by the threaded interconnection therebetween. This places the attached medical tubing 2270 into fluid communication with the spike 2000.

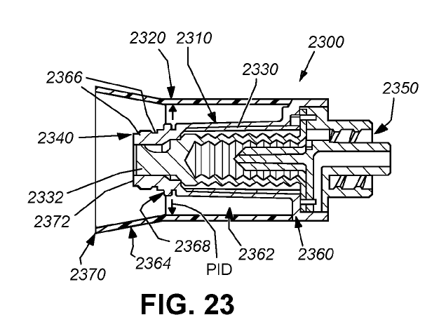

The disadvantages of a minimum fluid displacement coupling, as described above, can be addressed using a port guide in accordance with an embodiment of this invention. FIG. 23 shows an assembly 2300 including a minimum fluid displacement coupling 2310 enclosed within a port guide 2320 according to an illustrative embodiment. The coupling 2310 includes a housing 2330 that encloses a spring-biased plug 2332 that selectively seals the threaded female luer taper port 2340 against fluid flow with respect to the opposing male taper luer port 2350. The arrangement and function of the minimum fluid displacement coupling 2310 is similar or identical to the exemplary prior art coupling 700 as described above. However, the coupling can be constructed with a variety of alternate shapes and internal mechanisms according to alternate embodiments, and for the purposes of the illustrative embodiments, it is desired mainly that the coupling allow for a self-sealing coupling port at one end. As such, the opposing end can be integrally or unitarily connected to a fluid component, such as a stopcock, or the opposing end can be a removable threaded coupling (e.g. male coupling 2350) as shown. In this embodiment, the port guide 2320 is mounted against the base 2360 of the minimum fluid displacement coupling 2310 and defines a space 2362 between the inner wall of the port guide 2320 and the outer wall of the coupling 2310. Where the space is present, appropriate drain ports can be provided near the base 2360 and proximally spaced therefrom. In alternate embodiments, the space can be omitted, so long as the proximal region 2364 of the port guide defines a sufficient inner diameter PID to accommodate the outer diameter of the largest threaded sleeve or cannula to be received by the female port 2340 and its external thread 2366 external thread. The port thread 2366 ends at a distal shoulder 2368. The proximal region 2364 should maintain the sufficient inner diameter PID proximally of this shoulder 2368. In this embodiment, the proximal region flares outwardly in the proximal direction, thereby providing a funnel-like effect for an approaching cannula. The relative angle of the taper or flare with respect to the axial direction, and its particular geometric shape, are highly variable. Likewise, the distance that the proximal edge 2370 of the port guide 2320 extends beyond the proximal edge 2372 of the female port 2340 is variable. In an embodiment, a distance of extension between approximately 5 and 8 millimeters provides sufficient clearance for the shoulders of syringes and other male couplings while preventing inadvertent contaminating contact with the port 2340.

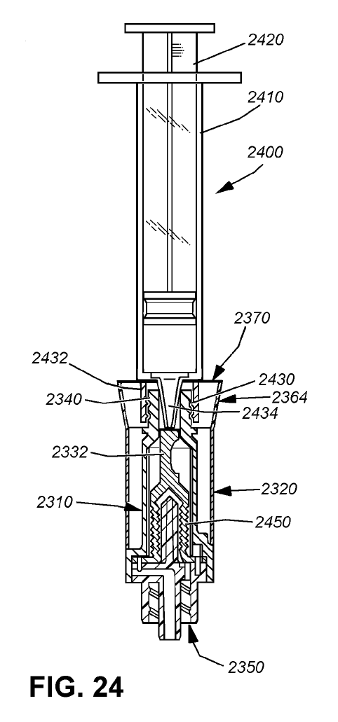

As depicted in FIG. 24, a small diameter syringe 2400 with a body 2410, plunger 2420 and distal cannula 2430 having an internal threaded sleeve 2432 and male luer taper coupling tip 2430 is twisted into full engagement with the female luer taper port 2340 of the assembly 2300. The coupling tip 2434 depresses the plug 2332 against the biasing force of its interconnected spring portion 2450, which is shown under compression. This allows fluid to pass through the coupling's housing and into the opposing port 2350. When the syringe 2400 is untwisted from the port 2340, the spring 2450 will bias the plug 2332 back into a sealed orientation, preventing fluid leakage therefrom. Meanwhile, the surrounding port guide 2320 prevents contamination of the port 2430 while providing an additional gripping surface for the practitioner to employ while twisting and untwisting the syringe or other connected fluid system component.

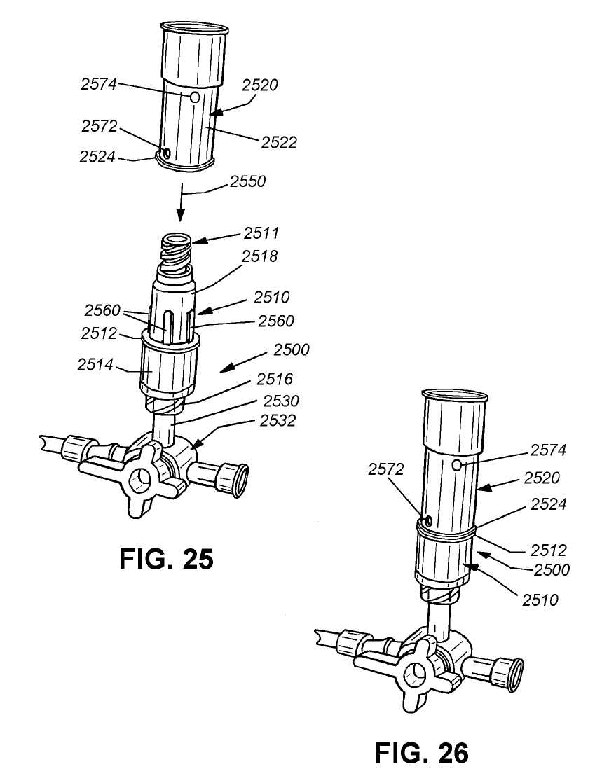

FIGS. 25 and 26 show an embodiment of an assembly 2500 consisting of a conventional minimum fluid displacement coupling 2510 with an attached port guide 2520. In this embodiment, the conventional coupling 2510 includes (in addition to a self-sealing threaded female luer taper port 2511) a base shoulder 2510 and larger diameter base section 2514 that terminates in a distal threaded male luer taper coupling 2516. In this example, the coupling 2516 is threadingly attached to the female luer taper coupling of port 2530 on a conventional stopcock 2532. This stopcock is attached to a medical fluid system as shown. The smaller diameter proximal portion 2518 of the exemplary minimum fluid displacement coupling 2510 receives (arrow 2550) the distal end 2522 of a port guide thereover. The fully assembled version of the assembly 2500 is shown particularly in FIG. 26 in which the distal edge 2524 of the guide 2520 engages the shoulder 2512 of the coupling 2510. In this example, the coupling's proximal portion 2518 includes gripping protrusions 2560 that generate a small cavity between the inner wall of the guide and the outer wall of the coupling in the adjacent region. Thus one or more distal drain ports 2572 (as well as more-proximal drain ports 2574) are provided. In alternate embodiments, the distal edge 2524 of the guide can be constructed to allow excess fluid to run past it (using notches or other passageways). The port guide 2510 of this embodiment can be a removable component, or can be permanently attached to the minimum fluid displacements coupling 2520 using adhesives, welding, fasteners, interengaging threads and the like. In illustrative embodiments the port guide can be formed together (e.g. co-molded) with a minimum fluid displacement coupling of any acceptable mechanism and shape.

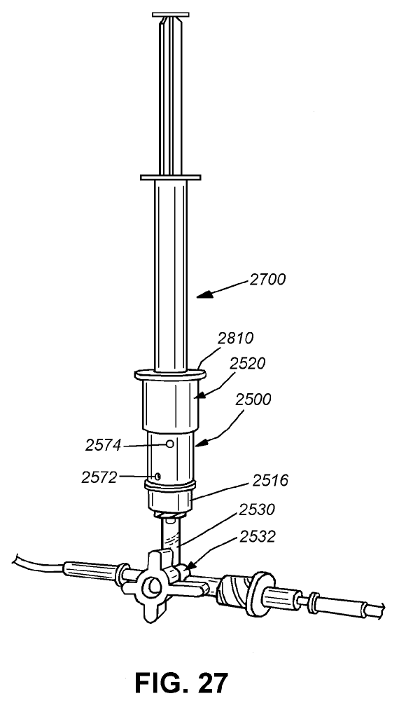

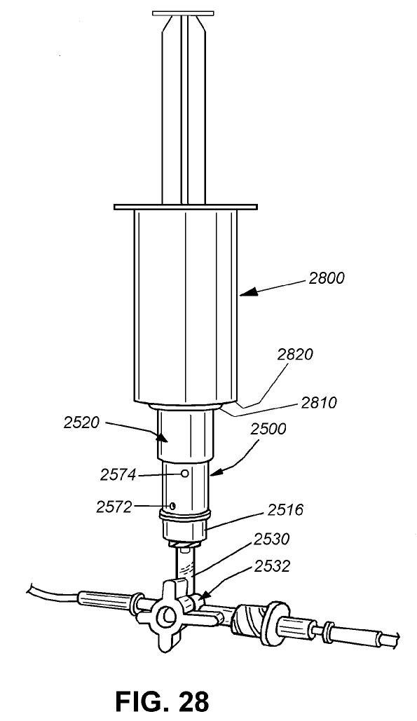

Briefly, as shown in FIG. 27, a small-diameter syringe or other fluid component is accommodated by the port guide and coupling assembly 2500 with reduced risk of contamination of the self-sealing female luer taper port (2510 in FIG. 25). Likewise, as shown in FIG. 28, the placement of the port guide's proximal edge 2810 relative to the port (2511) allows a syringe 2800 or other component with a standard cannula shape/dimension and a proximal component diameter greater than the port inner diameter to be accommodated. In this example the syringe shoulder 2820 resides out of interfering contact (or just barely in interfering contact) with the proximal edge 2810 of the guide 2520 when the syringe is twisted into full engagement with the port (2511).

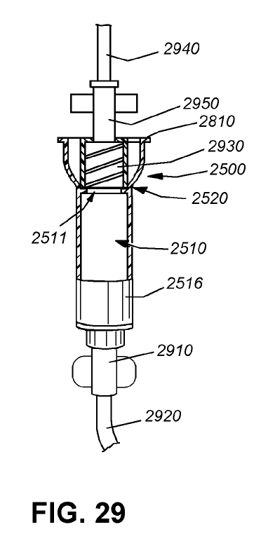

The minimum fluid displacement coupling 2510 and port guide 2520 of the illustrative assembly 2500 (or any other arrangement contemplated herein) can be interconnected to a variety of system components either integrally/unitarily (i.e. as a non-removable part of the component's structure), or as a selectively attachable/detachable component. FIG. 29 details one of a variety of possible interconnections in which the assembly 2500 can be employed. As shown, the male coupling 2516 of the assembly 2500 is mounted onto a female luer taper coupling 2910 on the end of a fluid tubing 2920. In alternate embodiments, the connection between the tubing (or other fluid system component) and the assembly can be a permanent connection or a luer slip-style connection. The opposing self-sealing threaded female luer taper coupling 2511 is threadingly attached to the internally threaded sleeve 2930 of the male luer taper connector of a fluid tubing 2940. Since the sleeve 2930 resides even with or slightly beneath the proximal edge 2810 of the port guide 2520 in this example, the practitioner tightens the coupling sleeve 2930 to the port 2511 by applying twisting force to the exposed proximal stem 2950 that is fixedly attached to the sleeve 2930 in this example. One or more grip wings 2960 can be optionally provided to the stem at a location spaced-apart from the proximal edge 2810. A variety of alternate mechanisms can be used to allow a shallow sleeve to be tightened onto the recesses port when surrounded by a port guide.

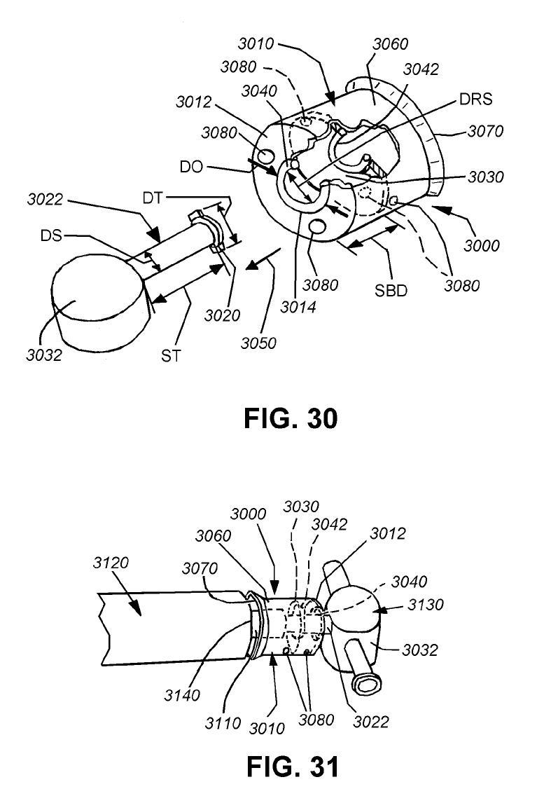

As described above, the port guide use in conjunction with a minimum fluid displacement coupling can be constructed as an attachable/retrofittable item for use with conventional non-shielded couplings. Likewise an attachable/retrofittable port guide for use with a conventional threaded female luer taper coupling port can be provided in accordance with the embodiment shown in FIGS. 30 and 31. Referring to the cutaway view of FIG. 30, the attachable port guide 3000 is constructed from any acceptable material, such as a transparent or translucent polymer. It defines a sidewall 3010 and a distal base 3012 with a central orifice 3014 having a diameter DO greater than the diameter DT of a conventional external thread 3020 of a conventional female luer taper port 3022. A similarly dimensioned circumferential bulkhead 3030 is located within the enclosure of the sidewall at an axially proximal spacing distance SBD that is less than the spacing ST along the stem 3022 between the stem's distal end (in this example the joint with the valve chamber 3032) and the distal side of the thread 3020. The difference between SBD and ST is sufficient to position the bulkhead 3030 remote from the thread so as to avoid interference with a fully engaged cannula (see FIG. 31). Notably, a central resilient support 3040 is respectively within the central orifice 3014 of the port guide's distal base 3012. Another resilient support 3042 of similar dimension is seated within a similar orifice within the bulkhead 3030. These resilient supports 3040, 3042 can be constructed from any flexible material, such as rubber, soft PVC, and the like. The supports 3040, 3042 can take the form of O-rings in an embodiment. In other embodiments the supports are flexible washers. In general, the supports 3040, 3042 are flexible enough to elastically deform as they are driven (arrow 3050) over the thread 3020 of the port stem 3022. The inner diameter DRS of each resilient support is approximately equal to or slightly smaller than the outer diameter DS of the stem 3022. In this manner, the supports engage and frictionally capture the stem, as shown in the assembled arrangement of FIG. 31. The proximal region 3060 (proximal of the bulkhead 3030) of the port guide 3000 an inner diameter sufficient to accommodate the outer diameter of a standard cannula with internally threaded male luer taper coupling 3110 (shown partially in phantom in FIG. 31) of a syringe 3120 or other fluid system component. All, or a portion of the proximal section 3060 can be proximally outwardly tapered or flared as shown.

In FIG. 31, the attached port guide is secured to the stem 3022 at two axially spaced apart locations thereby forming a secure, substantially wobble-free mounting with the guide distal base 3012 resting against the chamber 3022 of the exemplary stopcock 3130 or other fluid system component. As shown, the male coupling/cannula 3110 has sufficient clearance from the bulkhead 3030 to be fully engaged, and the syringe shoulder 3140 has clearance from the proximal edge 3070 of the guide 3000. The frictional coefficient of the resilient support, combined with the hoop stress induced by a slightly smaller diameter with respect to the stem, ensures that the guide 3000 remains axially fixed with respect to the underlying port in all orientations.

As described with respect to other embodiments herein, the port guide 3000 can be provided with drain ports 3080 along its distal base 3012, through the bulkhead 3030 and/or on the sidewall 3010 of the guide near the distal end and/or proximally above the bulkhead 3030—and/or at other appropriate locations.

It should be clear that the embodiment of an attachable or retrofittable port guide of FIGS. 30 and 31 has advantages in that the guide is easily attached to the port with a dingle distal motion, and that the user need not contact the interior of the guide or the exterior of the port during the attachment process-which is effectively a plug-together procedure. However, other techniques for attaching and securing an attachable or retrofittable port guide are expressly contemplated in alternate embodiments. For example, a port guide consisting of two separate molded halves can be brought together on the stem and adhered together using adhesives, etc. Likewise, a separate distal base member can be assembled on the port, and the sidewall section thereafter moved distally over the port and onto the assembled base. A variety of alternate mechanisms are also envisioned.

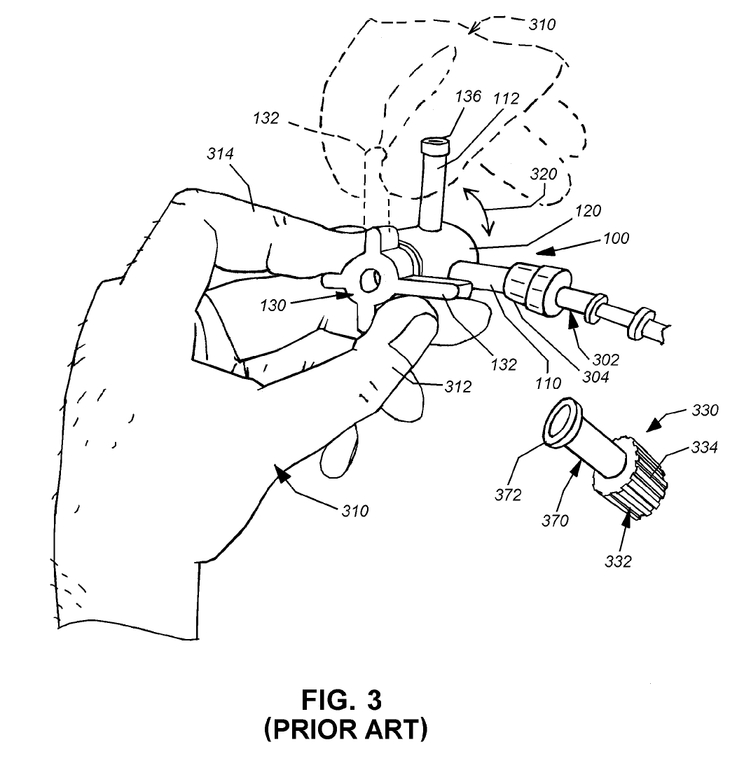

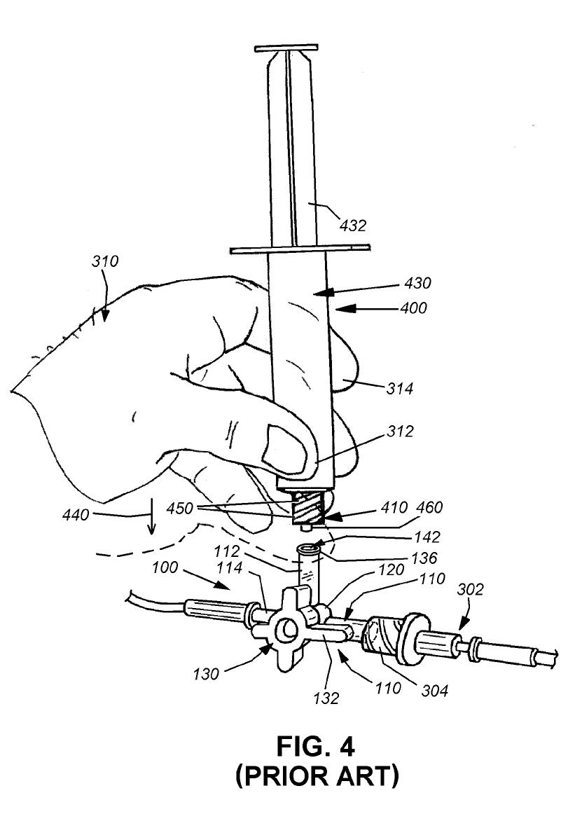

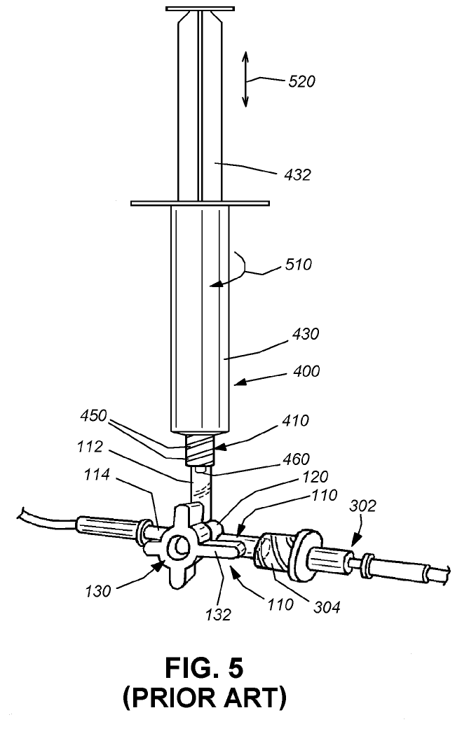

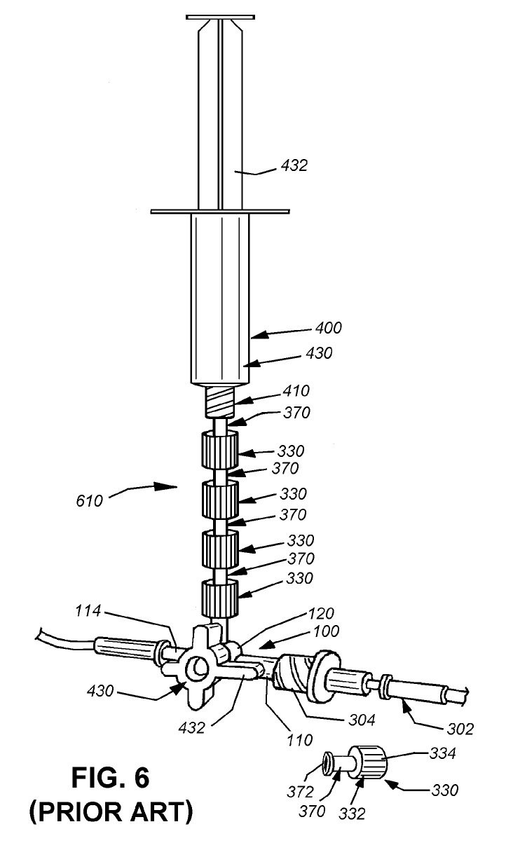

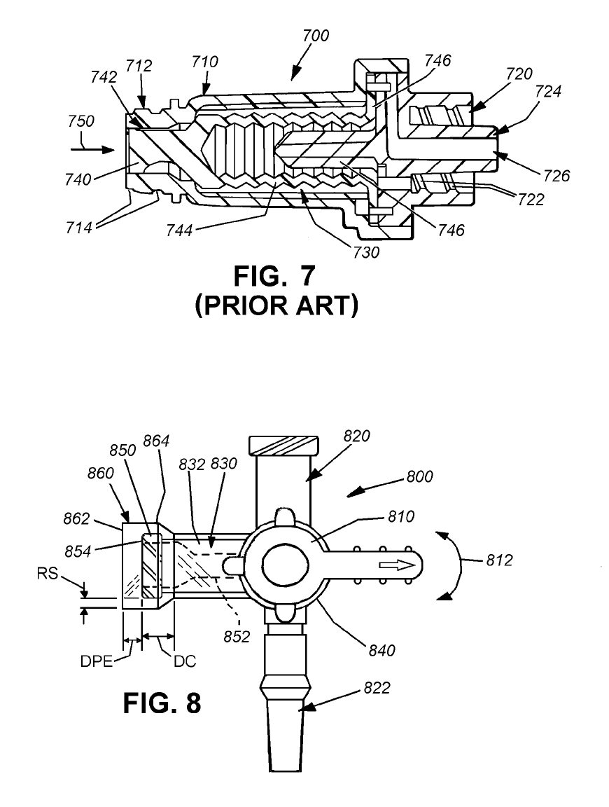

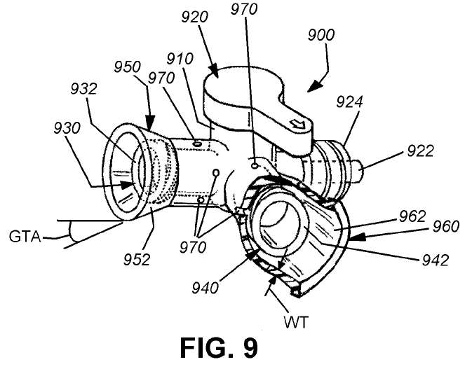

While not shown, other ports and port-like components can benefit from the port guide arrangement of the illustrative embodiments. For example, the dead-end cap 330 (FIG. 3) can be provided with a port guide that extends from the internally threaded sleeve proximally past (and surrounding) the stem 370 and thread end 372. In this manner the thread end cannot be contaminated while the cap 330 is being handled. In this manner further stacked caps, etc. that engage the thread 372 have reduced risk of contamination. Thus, for the purposes of the description, the stem 370 and thread 372 can be considered a female “port” to which the guide can be applied.

In early clinical studies it has been revealed that the use of a port guide on both a standard threaded female luer taper coupling and a minimum fluid displacement coupling has beneficial effects on the reduction of both port and effluent contamination when compared with unshielded ports. In such studies practitioners, using regular and established techniques, injected sterile saline into injection ports placed under the following conditions: (a) unshielded, (b) fitted with a port guide as described herein, (c) fitted only with an unshielded minimum fluid displacement coupling, and (d) fitted with a minimum fluid displacement coupling (clave) having an attached port guide as described herein. Microbiological culture samples were then taken from the lever, injection port and injection port-directed effluent to determine the rate of bacterial contamination associated with each type of injection port (a-d). Petri dishes were inoculated with the microbiological culture samples to evaluate the lever, injection port and port-directed effluent for sterility. The lever, which comes into contact with practitioner hands, represents an expected site of bacterial contamination (thus the high percentage of fluid system lever bacterial contamination). The lumen of the injection port and the port-directed effluent should ideally have no bacterial contamination. Thirty-six practitioners participated in the study, and results are reported as a percentage of practitioners whose levers, lumens or port-directed effluent were bacterially contaminated, comparing ports a-d. The results of the cultures are shown in the following table. By way of example: 28 of 36 practitioners contaminated the lever of the unshielded port (78%), and 6 of 22 practitioners contaminated the effluent (27%).

| | MICROBIOLOGICAL GROWTH | | |
|---|---|---|---|
| TYPE OF PORT | Lever | Lumen | Effluent |
| Unshielded Port | 78% (28/36) | 17% (6/36) | 27% (6/22) |
| Guide-Shielded Port | 89% (32/36) | 3% (1/36) | 0% (0/22) |
| Unshielded Clave | 78% (28/36) | 6% (2/36) | 18% (4/22) |
| Guide-Shielded Clave | 75% (27/36) | 8% (3/36) | 4% (1/22) |

Based upon the above results, it should be clear that the degree of microbiological contamination for the lumen, and importantly the degree of effluent contamination, is significantly reduced in both the port guide-shielded standard female stopcock port and the stopcock port with port-guide-shielded minimum fluid displacement coupling (clave) attached thereto. This reduction occurs despite relatively high contamination levels on stopcock levers for all stopcocks used in the test.

In summary, the illustrative port guide effectively reduces the risk of contamination to ports employed on a variety of fluid system components. It is applicable to both standard ports and those employing a clave. It renders the procedure of attaching a syringe or other device easier and allows the practitioner to grasp the region of the port more closely without the risk of contamination to the port lumen/orifice or surrounding locking structure (e.g. threads). It also ensures that the port remains untouched by non-sterile objects during follow-on use between injections/interface with the port.

The foregoing has been a detailed description of illustrative embodiments of the invention. Various modifications and additions can be made without departing from the spirit and scope of this invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. For example, while to port guide is shown as generally cylindrically shaped with a widened aperture and made of plastic/polymer, the port can be of different sizes and cross sectional shapes (e.g. polygonal, ovular, etc.), and constructed of different material (or combinations of materials), such as glass, polycarbonate, steel, resin, plastic, etc. Moreover, while the guide is located around a female port structure, it can be used in conjunction with a male coupling where appropriate or with a genderless coupling. In addition, while the ports are illustratively locking or slip-style luer taper ports, the guide can be adapted for use with other forms of medical fluid couplings such as those receiving needle injections. Accordingly, this description is meant to be taken only by way of example, and not to otherwise limit the scope of this invention.

What is claimed is:

1. A medical fluid coupling comprising:
a female port of a first medical fluid system component including a proximal port end constructed and arranged to sealingly engage a male port coupling; and
a port guide defining a sidewall that surrounds the female port and extends from a distal end of the female port to a proximal guide end, the proximal guide end being open to receive the male port coupling and located proximally at a spacing from the proximal port end, so as to prevent contaminating contact with the female port and aid in guiding the male port coupling into alignment and engagement with the proximal port of the female port.

2. The medical fluid coupling as set forth in claim 1 wherein the female port comprises a female luer taper port and the male port comprises a male luer taper port.

3. The medical fluid coupling as set forth in claim 2 wherein proximal port end includes an external locking thread and the male port includes an internally threaded sleeve, surrounding a luer taper connector tip constructed and arranged to threadingly engage the external thread.

4. The medical fluid coupling as set forth in claim 3 wherein the female luer taper port and the male luer taper port are each defined by at least one of an ANSI and an ISO standard.

5. The medical fluid coupling as set forth in claim 3 wherein the female port includes a housing on a distal region thereof comprising a minimum fluid displacement coupling and the proximal port end includes a movable self-sealing plug therein.

6. The medical fluid coupling a set forth in claim 1 wherein the first medical fluid system component is at least one of a tubing, a stopcock, an IV bag spike, a minimum fluid displacement coupling, an adapter, a fluid monitoring device, a fluid pumping device, a fluid handling device, and a dead-end cap.

7. The medical fluid coupling as set forth in claim 1 further comprising one or more drain ports located in the sidewall of the port guide.

8. The medical fluid coupling as set forth in claim 1 wherein the port guide defines a discrete structure that is constructed and arranged to be attachable to the port.

9. The medical fluid coupling as set forth in claim 8 wherein the port guide includes a circumferentially enclosed proximal portion and a distal portion having a pair of axially spaced apart resilient supports with an inner diameter approximately equal to or slightly smaller than an outer diameter of a stem of the female port, constructed and arranged to elastically pass over a proximal end of the female port and frictionally capture the distal stem of the female port during attachment to the female port.

10. The medical fluid coupling as set forth in claim 8 wherein the female port includes a housing on a distal region thereof comprising a minimum fluid displacement coupling and the proximal port end includes a movable self-sealing plug therein, and wherein the port guide includes a distal portion constructed and arranged to slidably engage upon the housing.

11. The medical fluid coupling as set forth in claim 1 wherein the male port comprises at least one of a syringe internally threaded locking cannula, a fluid tubing end coupling, an adapter coupling and a dead-end cap coupling.

12. The medical fluid coupling as set forth in claim 1 wherein the port guide defines, at a proximal region thereof, an outward taper in the proximal direction.

13. A port guide for use with a female externally threaded luer taper medical fluid coupling port comprising:
a sidewall that surrounds the port and extends from a distal end of the port to a proximal guide end, the proximal guide end being open and having an inner diameter constructed and arranged to receive a male port coupling having an internally threaded sleeve, and the proximal guide end being located proximally at a spacing from the proximal port end, so as to prevent contaminating contact with the port.

14. The port guide as set forth in claim 13 wherein the sidewall defines a discrete structure that is constructed and arranged to be attachable to the port.

15. The port guide as set forth in claim 14 wherein the port guide includes a circumferentially enclosed proximal portion and a distal portion having a pair of axially spaced apart resilient supports with an inner diameter approximately equal to or slightly smaller than an outer diameter of a stem of the female port, constructed and arranged to elastically pass over a proximal end of the port and frictionally capture the distal stem of the female port.

16. The port guide as set forth in claim 14 wherein the port includes a housing on a distal region thereof comprising a minimum fluid displacement coupling and the proximal port end includes a movable self-sealing plug therein, and wherein the sidewall includes a distal portion constructed and arranged to slidably engage upon the housing.

17. A medical fluid coupling comprising:
a minimum fluid displacement coupling defining a housing with a proximal externally threaded female port having a self-sealing plug adapted to selectively allow fluid flow into the housing when biased distally by an ANSI or ISO standard male internally threaded taper luer cannula; and
a port guide sidewall that surrounds the female port and extends proximally to a proximal edge a predetermined spacing distance from the proximal end of the female port, the port guide sidewall defining a radial spacing in a proximal region thereof that allows the male cannula to threadingly engage the female port free of interference from the port guide sidewall.

18. The medical coupling as set forth in claim 17 wherein the housing includes a distal male internally threaded taper luer coupling.

19. The medical coupling as set fort in claim 18 wherein the port guide sidewall includes at least one drain port to allow excess fluid to drain from between the housing and the port guide sidewall.

20. The medical coupling as set forth in claim 18 wherein the port guide sidewall is constructed and arranged to slidably engage the housing by passing a distal end of the port guide sidewall over the female port and into seating engagement with distal base of the housing.
